(19) United States
(12) Patent Application Publication
(10) Pub. No.: US 2019/0083565 A1
(43) Pub. Date: Mar. 21, 2019

(54) WATER SOLUBLE FORMULATION (LIQUID) OF CURCUMINOIDS AND PROCESS THEREOF

(71) Applicant: MEDCLEAR HEALTHCARE PRIVATE LIMITED, Hyderabad (IN)

(72) Inventors: Ajay Sunil Kiran Kumar Potti, Hyderabad (IN); Krishna Veni Vadlamudi, Khammam (IN)

(21) Appl. No.: 16/088,451

(22) PCT Filed: Mar. 7, 2017
(86) PCT No.: PCT/IB2017/051315

§ 371 Date: Sep. 26, 2018

(30) Foreign Application Priority Data
Apr. 7, 2016 (IN) 201641012270

(51) Int. Cl. A61K 36/9066; A61K 47/20; A61K 47/26; A61K 47/10; A61K 47/32; A61K 47/38

(52) U.S. Cl. CPC A61K 36/9066; A61K 47/20; A61K 47/38; A61K 47/10; A61K 47/32; A61K 47/26

(57) ABSTRACT

Exemplary embodiments of the present disclosure are directed towards a water soluble viscous formulation comprising of; an active ingredient comprising of an unprocessed, crystalline herbal extract of curcuminoid, whereby the amount of active ingredient incorporated ranges between 0.5% to 10% weight by volume of the total composition; a surfactant comprising of a surfactant, whereby the amount of surfactant incorporated ranges between 0.01% to 10% weight by volume of the total composition; a vehicle comprising of hydrophilic vehicle, whereby the amount of the hydrophilic vehicle incorporated ranges between 10% to 90% weight by volume of the total composition; and a hydrophilic polymer comprising of a water soluble polymer, whereby the hydrophilic polymer ranges between 1% to 40% weight by volume of the total composition.

### TECHNICAL FIELD

The present disclosure generally relates to the field of processed pharmaceutical and nutraceutical additives. More particularly, the present disclosure relates to enhanced solubility leading to increased bioavailability of curcuminoids for effective usage in applicable areas.

### BACKGROUND

Curcumin (1,7-bis [4-hydroxy-3methoxyphenyl]-1,6-heptadiene-3,5-Dione) is the principal curcuminoid of turmeric, which is a member of the ginger family (Zingiberaceae). Curcuminoid is an active ingredient in the traditional herbal remedy and dietary spice turmeric (Curcuma longa). Curcuminoid is obtained by extraction with solvent from the rhizome of Curcuma longa dried, and the extract must be separated from the flavourings i.e purification by crystallisation. It is accompanied by small amounts of its demethoxy- and bis-demethoxy-derivatives. As a group these compounds and their derivatives for pharmaceutical use are referred to as curcuminoids.

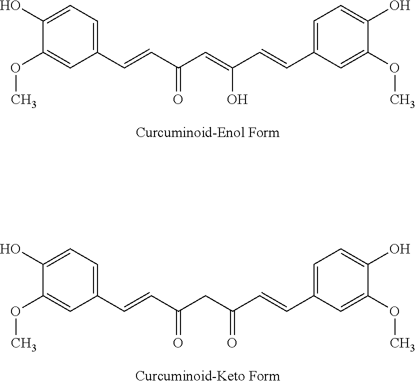 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

There have been recent evidences through studies conducted on the use of curcuminoids as dietary supplement in treating a variety of clinical disorders. Medicinal use of curcuminoids have been limited because the compound is virtually insoluble (dissolution of only ca. 600 ng/mL) in water at acidic and physiological pH and hydrolyzes rapidly at alkaline pH. Plasma concentrations from high human oral doses (8-12 g/day) of curcuminoids are just in the nano molar range.

Various clinical trials have shown that curcuminoids exhibit very poor bio-availability. Major reasons contributing to the low plasma and tissue levels of curcuminoids appear to be due to poor water solubility, poor absorption, rapid metabolism, and rapid systemic elimination.

Despite the lower bio-availability, therapeutic efficacy of curcuminoids against various human diseases, including cancer, Alzheimer's disease, cardiovascular diseases, diabetes, arthritis, neurological diseases and Crohn's disease, has been documented.

Curcuminoids are water insoluble (Solubility: 0.6 mcg/ml), so application in aqueous based foods, beverages is not feasible. Moreover, due to insolublity in water, absorption of curcuminoids is very low. So improvement in solubility of curcuminoids in water is desirable to get wide range of benefits. Disadvantages of existing products in market are explained below:

Most of the products (Capsules & Tablets) are required to take up more doses up to 1-2 g/day due to low bioavailabilty.

Some of the products are in nano form, but still in water dispersible form which may hinder absorption. Some products are water soluble powder form, but not feasible for some applications as turbidity issues are not being addressed due to improper solubility.

Chinese patent number CN100352430C discloses curcuminoids in the form of micro emulsion formulation and a preparation process for the same. The formulation consists of curcuminoids, surface active agents, surfactants, oil phase and a solid sorbent composition and can be made into capsules or granules for oral administration. Post entering the body it is believed that under the influence of self-micro emulsifying gastrointestinal fluid forming droplets the nano particle size of 100nm or less, one can increase the solubility of curcuminoid and promote its absorption in the gastrointestinal tract to improve bioavailability.

WO 2013171270 discloses gastro resistant formulations of curcuminoid combined with absorption promotors (chitosan, black pepper extract, N-acetylcysteine, grapefruit extract). Said formulations are not controlled-release or extended-release formulations.

US 20140161915 discloses a process for solubilizing and stabilizing curcuminoid in an aqueous solution, comprising: (a) providing a quantity of water; (b) providing over 0.008 mg curcuminoids per mL water; (c) optionally providing at least 0.01 ml lower alcohol per ml of water; (d) optionally providing at least 0.01 ml polyalkyleneoxide per ml of water; (e) 6 combining the water, curcuminoid and optional lower alcohol in any order and optionally with mixing; and (f) heating the combined water, curcuminoid and optional lower alcohol to a temperature in excess of 100%, optionally at a pressure exceeding 1 atmosphere; wherein steps (a), (b) and (c) may be done in any order, and wherein the process includes at least one of combining the lower alcohol or optional polyalkyleneoxide in step (e) and applying a pressure exceeding 1 atmosphere in step (f).

US 2011/0274809 disclose a method for masking curcuminoid flavour which comprises mixing curcuminoids with a modified starch.

CN101361713B discloses the invention relates to a curcuminoid nano crystal formulation and a preparation thereof. The nano crystal can increase the solubility of the curcuminoid, promote the absorption thereof in gastrointestinal tract and improve bioavailability.

WO2014/111956 discloses a sustained release curcuminoid composition comprising a bioavailable curcuminoid composition and a release rate controlling excipient.

CN103442703B/U.S. Pat. No. 9,259,401B2 discloses water-soluble composition having enhanced bioavailability useful for the treatment of depression comprising curcuminoid, a synergistic combination of at least one anti-oxidizing agent, a hydrophilic carrier and fats.

In the light of aforementioned discussion there exists a need of enhanced water solubility in turn increasing the bio-availability of curcuminoid in order to bring curcuminoid and its derivatives to the forefront of therapeutic agents for treatment of various diseases.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An exemplary objective of the present disclosure is, to provide water soluble formulation of curcuminoids, preferably in liquid form without using any organic solvents to enhance bioavailability of curcuminoids in vivo.

Another objective according to an exemplary embodiment of the present disclosure is, to provide an amorphous form of water soluble formulation of curcuminoids in order to achieve higher solubility resulting in higher bioavailability.

Another objective according to an exemplary embodiment of the present disclosure is, to provide water soluble formulation of curcuminoids, to facilitate easy miscibility with water, aqueous based beverages, foods, and dietary supplements and to eliminate problem of sedimentation which is associated with dispersible formulations in beverage application with existing products in the market.

Yet another objective according to an exemplary embodiment of the present disclosure is, to provide water soluble formulation of curcuminoids, using excipients having E-Number/generally recognised as safe (GRAS) status that ensures safety of human beings for long term administration.

Further objective of the present invention is to provide water soluble formulation of curcuminoids, preferably in liquid form with easy process to reduce the cost and time of the process.

An exemplary aspect of the present subject matter is, to provide a water soluble formulation of curcuminoids and methods employed thereof.

Another exemplary aspect of the present subject matter is to provide water soluble formulation of curcuminoids in an amorphous form and high storage stability.

Another exemplary aspect of the present subject matter is to provide water soluble formulation of curcuminoids capable of acting as natural colourants for consumables and being non-carcinogenic.

Yet another exemplary aspect of the present subject matter is to provide water soluble formulation of curcuminoids required in low dosage due to enhanced bioavailability.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a flow diagram illustrating the method employed for the preparation of water soluble formulation of curcuminoids, according to an exemplary embodiment of the present disclosure.

FIG. 2 is a graphical representation depicting the solubility pattern of natural curcuminoids to that of water soluble formulation of curcuminoids, according to an exemplary embodiment of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

The present disclosure provides water soluble formulations of Curcuminoids comprising of;
Turmeric extracts (Curcuminoid 95%) (Curcuma Longa) (Preferably, mixture of curcuminoid, demethoxy curcuminoid, and bis demethoxy Curcuminoid);
A Surfactant and/or Emulsifier;
At least one vehicle
A Hydrophilic Polymer
A Preservative
An Acidulant
Overages

The terms “curcuminoid” and “curcuminoids” used herein refers to one or a mixture of curcuminoid, demethoxy curcuminoid, and bis demethoxy curcuminoid.

The Surfactants and/or Emulsifiers used are selected from sodium lauryl sulfate, Polysorbate 80 (Tween 80), docusate sodium or mixture thereof and the like without limiting the scope of the disclosure. The vehicles incorporated may include but not limited to Purified Water, Polyethylene Glycol, preferably PEG 400, and Propylene Glycol, Polysorbate 80 or mixture thereof. The hydrophilic polymers used are selected from hydroxy propyl methyl cellulose, polyvinyl pyrollidone, Copovidone (vinylpyrrolidone-vinyl acetate copolymer) or mixture thereof and the like without limiting the scope of the disclosure.

The preservatives used are selected from sodium benzoate, benzoic acid, potassium sorbate, sodium sorbate or mixture thereof and the like without limiting the scope of the disclosure. The acidulants used may include but not limited to citric acid, acetic acid, lactic acid, malic acid, tartaric acid or mixture thereof. The overages are added which may be less than 1% of the total formulation.

An exemplary embodiment for the composition for the manufacture of water soluble formulation of curcuminoids is represented in the following table:

| S. No | Ingredient | Role | Range | % w/v | Qty/Batch | |
|---|---|---|---|---|---|---|
| 1 | Turmeric Extract | Active | 0.5-6 | 5.4 | 5.4 | Kg |
| | (Curcuminoid 95%) | ingredient | | | | |
| | (Curcuma Longa) | | | | | |
| 2 | PolyethyleneGlycol | Vehicle | 10-45 | 28 | 28 | Kg |
| | 400(PEG 400) | | | | | |
| 3 | Propylene Glycol | Vehicle | 10-60 | 19.85 | 19.85 | Kg |
| 4 | Polyvinylpyrollidone | Hydrophilic | 10-25 | 19 | 19 | Kg |
| | (PVP K 30) | polymer | | | | |
| 5 | Polysorbate 80 | Surfactant | 2-15 | 8 | 8 | Kg |
| | (Tween 80) | | | | | |
| 7 | Citric Acid | Acidulant | 0.1-2 | 0.5 | 0.5 | Kg |
| 8 | Potassium Sorbate | Preservative | 0.01-0.2 | 0.1 | 0.1 | Kg |
| 9 | Sucralose | Readily | 0.05-1.0 | 0.1 | 0.1 | Kg |
| | | soluble | | | | |
| | | sweetner | | | | |
| 10 | Orange flavour | Flavourant | 0.01-1 | 0.05 | 0.05 | Kg |

| | | | | | |
|---|---|---|---|---|---|
| 11 | Purified Water | Vehicle | 1-30 | QS | Quantity |
| | | | | to 100 | Sufficient |
| | | | | | to 100 |
| | | | | | Litres |

QS = Quantity Sufficient

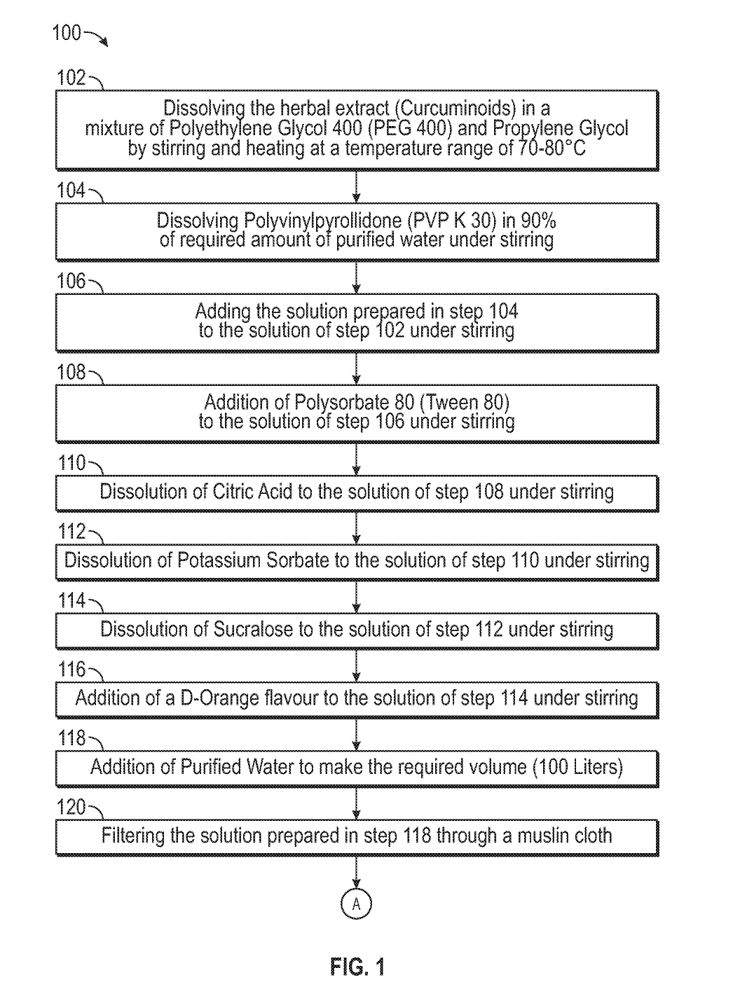

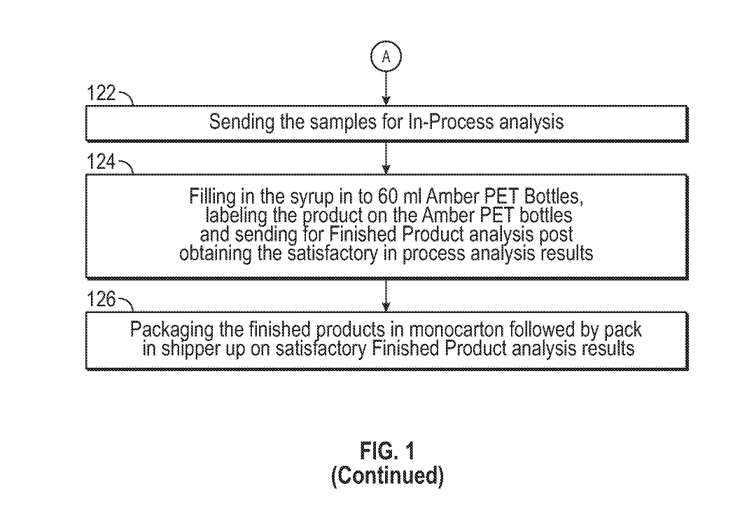

Referring to FIG. 1 is a flow diagram 100, illustrating the method employed for the preparation of water soluble formulation of curcuminoids, according to an exemplary embodiment of the present disclosure. The method starts at step 102 by dissolving the herbal extract (Curcuminoids) in a mixture of Polyethylene Glycol 400 (PEG 400) and Propylene Glycol by stirring and heating at a temperature range of 70-80° C. Further, Polyvinylpyrollidone (PVP K 30) is dissolved in 90% of required amount of purified water under stirring at step 104. Further at step 106, the solution prepared in step 104 is added to the solution prepared in step 102. Further, addition of Polysorbate 80 (Tween 80) to the solution of step 106 under stirring takes place at step 108. Dissolution of Citric Acid to the solution of step 108 under stirring is done at step 110. At step 112, dissolution of Potassium Sorbate to the solution of step 110 under stirring takes place. Dissolution of Sucralose to the solution of step 112 under stirring is done at step 114. Addition of a D-Orange flavor to the solution of step 114 under stirring is done at step 116. At step 118, the addition of purified water to make the required volume (100 Liters) is done. Further, the solution prepared in step 118 is filtered through a muslin cloth at step 120. The prepared samples are sent for In-Process analysis at step 122. At step 124, filling in the syrup in to 60 ml amber PET Bottles, labeling the product on the amber PET bottles and sending for finished product analysis post obtaining the satisfactory in process analysis results is done. Finally at step 126, packaging the finished products in monocarton followed by pack in shipper is done upon satisfactory finished product analysis results.

The water soluble formulation of curcuminoids is in the form of viscous liquid can be easily soluble/miscible in aqueous based beverages, foods, cosmetics and pharmaceutical products. The water soluble formulation of curcuminoids is in the form of viscous liquid enabled to provide a light yellow to orange shades as colorant.

The water soluble formulation of curcuminoids in the form of viscous liquid when dissolved provides clear solutions in water, with almost nil undissolved particles settled at the bottom.

The method employed for the preparation of water soluble formulation of curcuminoids transforms the crystalline form of curcuminoids to amorphous form which is soluble in water.

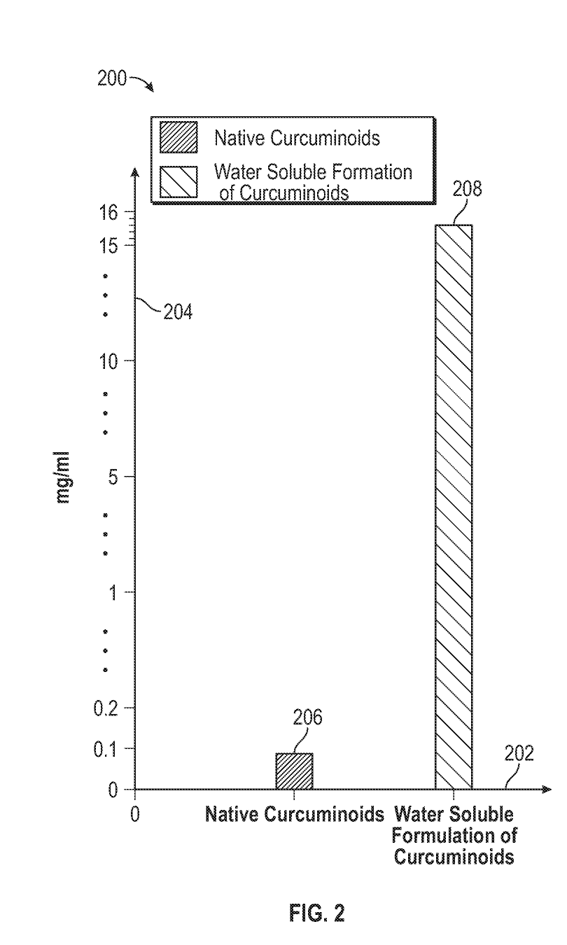

Referring to FIG. 2 is a graphical representation 200, depicting the solubility pattern of natural curcuminoids to that of water soluble formulation of curcuminoids, according to an exemplary embodiment of the present disclosure. The X-axis 202 represents both the products; the native curcuminoid which is the unprocessed naturally occurring curcuminoid and the water soluble formulation of curcuminoids subjected to graphical analysis for their respective solubility patterns. The Y axis 204 represents the solubility denoted as per milligram of the product under analysis to per millilitre of water i.e mg/ml. Upon experimentation it was found that the native curcuminoid's dissolution rate was <0.1 mg/ml, represented as a bar 206. Similarly when the water soluble formulation of curcuminoids was subjected to experimentation then, its dissolution rate was found to be 15.6 mg/ml, represented as a bar 208.

The difference in the solubility pattern of native curcuminoid and water soluble formulation of curcuminoids refers to higher availability of water soluble formulation of curcuminoids. This result in higher bioavailability making the water soluble formulation of curcuminoids appropriate for pharmaceutical and nutraceutical compositions.

The increase in bioavailability leads to faster action of the drug at the site of action thus leading to more effective treatment for any specific medical condition. The higher solubility leads to clarity in the solution when mixed with water thus eliminating turbidity leading to better usage in food processing industries like beverages, aqueous based foods and the like without limiting the scope of the disclosure.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. A water soluble viscous formulation comprising of;
an active ingredient comprising of an unprocessed, crystalline herbal extract of curcuminoid, whereby the amount of active ingredient incorporated ranges between 0.5% to 10% weight by volume of the total composition;
a surfactant incorporated ranges between 0.01% to 10% weight by volume of the total composition;
a vehicle comprising of hydrophilic vehicle, whereby the amount of the hydrophilic vehicle incorporated ranges between 10% to 90% weight by volume of the total composition;
and
a hydrophilic polymer comprising of a water soluble polymer, whereby the hydrophilic polymer ranges between 1% to 40% weight by volume of the total composition.

2. The formulation of claim 1, wherein the surfactant is further selected from at least one of: sodium lauryl sulphate; Docusate sodium; Polysorbate 80; and their corresponding mixtures.

3. The formulation of claim 1, wherein the hydrophilic vehicle is selected from the group consisting of: propylene glycol; polyethylene glycol; polysorbates; glycerin; purified water; and their corresponding mixtures.

4. The formulation of claim 1, wherein the hydrophilic polymer is selected from the group consisting of: Povidone (Polyvinylpyrrolidone); Co-Povidone; Hydroxymethylcellulose; and their corresponding mixtures.

5. A method for preparing water soluble viscous curcuminoids formulation, comprising of;
(i) dissolving curcuminoids in hydrophilic vehicles under heating temperature 70-80° C.
(ii) dissolving hydrophilic polymer in hydrophilic vehicle, preferably purified water.
(iii) Addition of step (ii) solution in to step (i) solution under stirring
(iv) Addition of surfactant, followed by acidulant, preservative, sweetener and flavor.

6. The method of claim 5, wherein the crystalline form of curcuminoid is converted in to amorphous form of curcuminoid.

7. The method of claim 5, wherein the solubility of curcuminoid is greater than unprocessed herbal extract of curcuminoid.

8. The method of claim 5, wherein the bioavailability of curcuminoid is greater than unprocessed herbal extract of curcuminoid.
